(19) United States
(12) Patent Application Publication
(10) Pub. No.: US 2003/0163204 A1
(43) Pub. Date: Aug. 28, 2003

(54) Stent

(76) Inventor: Gerald Henner Rix, Perth (GB)

Correspondence Address: DRINKER BIDDLE & REATH, ONE LOGAN SQUARE, 18TH AND CHERRY STREETS, PHILADELPHIA, PA, 19103-6996, US

(21) Appl. No.: 10/296,670

(22) PCT Filed: May 25, 2001
(86) PCT No.: PCT/GB01/02323

(30) Foreign Application Priority Data
May 26, 2000 (GB) 0012764.7

(51) Int. Cl. A61F 2/04

(52) U.S. Cl. 623/23.7; 604/8

(57) ABSTRACT

There is provided an indwelling ureteral (ureteric) stent comprised of a hollow flexible tube with an upper end section, a substantially straight middle section and a lower end section. The upper and lower sections are preferably coiled. The coiled upper section has a diameter between 1 and 2.5 cm which retains the upper section of the stent in the kidney, and has perforations in the surface to allow drainage of urine from the kidney into the tube. The lower coiled section of the stent is G-shaped. The tip of this lower section assumes the horizontal portion of the G shape and contains an integral valve. The integral valve maintains an open flow of urine from the kidney to the bladder, but prevents the reflux of urine into the kidney during bladder contraction. The stent further comprises a small cuff or series of studs behind said valve against which a stent pusher may rest.

The present invention relates to an indwelling ureteral (ureteric) stent which exhibits improved anti-reflux properties and which also reduces bladder irritation.

Ureteral stents are used in endo-urological intervention on a daily basis to allow drainage of urine from the kidneys to the bladder in instances of actual or potential ureteral obstruction. Such instances include ureteral injury due to trauma, obstructive uropathy such as kidney stones, and following surgery in the upper or lower urinary tracts.

Generally, stents are comprised of a hollow tube made of flexible material, of length varying from 25-35 cm with an external diameter from about 1.5-3 mm and an internal diameter of about 0.5-2 mm. Both ends are curled, forming spirals which produce an ‘O’ shape at each end of the stent. This allows the upper end to be retained within the kidney and the lower end within the bladder, thus preventing movement after placement. The flexibility of the comprising material allows the stent to conform to any curves of the ureter and also allows placement and removal through narrow urological instruments placed by means of the urethra. Currently the commonest form of stent used is known as a Double J Stent, or Double Pigtail Stent.

There are several problems for the patient associated with the use of the stents. Specifically, these are that during voiding of the bladder, the increased intravesical pressure, which induces evacuation of the bladder, can result in a back flow or reflux of urine. The hollow tube construction allows urine to pass up the stent producing pressure in the kidney as the bladder contracts during urination. These events are known as reflux.

Urine passing from the kidney to the bladder is sterile. If however, the urine becomes contaminated in the lower urinary tract with infection by pyrogenic organisms, then reflux of this urine may result in the development of sepsis, which can damage the kidney and also have potentially lethal consequences for the patient. The risk of sepsis following the employment of an indwelling stent between the kidney and the bladder, means that there is a need to provide a ureteral stent which will maintain an open flow of urine from the kidney to the bladder, while also inhibiting the reflux of urine to the kidney.

Further, during bladder evacuation, the stent may retract into the ureteral orifice. This upwards migration of the stent is seen with many stents of the mono J stent type, wherein the lower end of the stent doesn't have a curl.

A further problem associated with the use of stents is that the lower coil irritates the bladder by touching its lining. This is usually caused by the volume of material comprising the lower coil as well as the tip of the lower coil digging into the bladder lining.

Presently in the field, there are a number of stents which try to overcome the problems associated with the use of such devices, these are outlined below:

Anti-Reflux Stents

An article by Ahmadzadeh (Stenting the Urinary System. D Yachia. ISBN 1899066829) discloses a Double Pigtail Stent with a transparent thin walled segment made of polyurethane which is designed to lie at the junction between the ureter and the bladder i.e. at the vesico-ureteric junction. The floppy polyurethane walls would co-apt with vesical pressure rise preventing reflux. They would also allow the slit like ureteric orifice, which is a natural valve, to remain closed during intra-vesical pressure rises, which is how reflux is prevented in the normal healthy ureter and bladder.

U.S. Pat. No. 5,019,102 discloses a valve system comprising two thin transparent membranes forming a bag open at the distal end attached to the lower end of an ordinary stent and again as the pressure rises within the bladder these are pressured together preventing reflux of fluid.

Conversely when urine needs to be excreted, they open out allowing fluid drainage into the bladder.

U.S. Pat. No. 564,783 teaches of a Double J Stent with a closed lower portion which does not allow urine to drain up or down it and therefore prevents reflux. The lower end also has a small side hole into which the tip of the lower end curls back into after stent placement this being aided by two magnets.

Stents to Reduce Bladder Irritation

U.S. Pat. No. 5,141,502 discloses a stent with a helical upper end and a lower end made of a softer, non-irritating material but containing a cuff at the level of the vesico-ureteric junction, which allows placement over a guide wire.

A stent with a softer coil at the lower end bonded on to reduce bladder irritation is described in U.S. Pat. No. 4,931,037.

International Patent Application No WO 9717094 teaches of a stent with a lower portion which tails off into a thinner flexible region whose small diameter reduces bladder irritation and also does not push open the vesico-ureteric junction to such an extent, but which is not hollow so no longer acts as a channel for urine drainage either.

It is an object of the present invention to provide an improved indwelling ureteric stent to provide drainage between the kidney and the bladder. It is a further object of the invention to prevent the reflux of urine from the bladder into the kidney, thereby preventing flank pain associated with voiding and also the passage of infected urine in the lower urinary tract into the kidney where this could cause damage to the upper urinary tract. A further aim of the present invention serves to reduce the irritation of the bladder, which is associated with the use of stents.

According to a first aspect of the present invention there is provided an indwelling ureteral stent constructed of flexible material which comprises a hollow elongated tubular body, said hollow elongated tubular body comprising an upper end section, a substantially straight middle section and a lower end section wherein the tip of the lower end section of the stent comprises a valve which permits the hollow body to be in an open or a closed position wherein the valve is an integral part of the flexible material comprising the stent.

More preferably the valve is a bicuspid valve having two leaflets or a tricuspid valve having three leaflets.

Most preferably the valve is a bicuspid valve.

In a preferred embodiment the valve is provided through the moulded interlay of the flexible material such that in the closed position at rest the leaflets of the valve lie flat against each other providing a seal which prevents urine passing up the stent.

A second aspect of the present invention relates to an indwelling ureteral stent constructed of flexible material which comprises a hollow elongated tubular body, said hollow elongated body comprising an upper coiled section, a substantially straight middle section, and a lower end section wherein the lower section forms a closed or substantially closed loop, such that in use the tip of the end section of the stent is not exposed and cannot contact the bladder lining.

Preferably the stent also comprises a valve as described herein.

Preferably the lower end section is “G” shaped or spherically shaped such that in use the tip of the end section will not contact the lining of the bladder.

Preferably the upper section comprises a coil, said coil including flexible material between 6 to 15 cm of flexible material coiled once or twice upon itself, said coil having a diameter between 1 and 2.5 cm.

Preferably the lower section comprises a coil, said coil including a flexible material wherein said material is coiled thus forming an “O” or a “G” shape with a diameter of between 0.5-2 cm and wherein the tip of the stent rests within the coil and therefore, in use does not contact the bladder lining.

More preferably the lower section is formed into a “G” shape such that the tip of the stent assumes the horizontal portion of the G shape.

A third aspect of the present invention relates to an indwelling ureteral stent constructed of flexible material which substantially comprises a hollow elongated tubular body, said hollow elongated tubular body comprising an upper end section, a substantially straight middle section and a lower end section wherein the flexible material decreases in external diameter from the upper end section to the lower end section such that there is maximum drainage in the upper urinary tract and minimum irritation in the lower urinary tract.

Preferably the stent also comprises a valve as described herein the lower end shaped as described herein to prevent contact of the valve in the tip with the bladder lining.

More preferably the flexible material is tapered in diameter towards the lower end, such that the lower third of the substantially straight middle section and the totality of the lower section are of a reduced diameter.

A fourth aspect of the present invention relates to an indwelling ureteral stent constructed of flexible material which comprises a hollow elongated tubular body, said hollow elongated tubular body comprising an upper end section, a substantially straight middle section and a lower end section, wherein the stent further comprises at least one projection against which a stent pusher may rest.

Preferably the stent also comprises a valve as described herein and/or at least one end of the stent is shaped as described herein to prevent contact of the tip with the bladder lining.

Preferably the stent is tapered as described herein.

Preferably the projection(s) form a cuff.

Preferably the projection(s) consist of a plurality of studs.

According to each aspect of the invention the flexible material of the stent may comprise any composition which forms a hollow tube.

The flexible material may have a cylindrical cross section.

Alternatively the flexible material may have any shape of cross section either throughout it's whole length or in one section alone, such as in the lower third alone, including a spiral, a star or an oval, especially wherein said shape facilitates drainage on the outer surface or accommodation to the natural contours of the urinary tract preventing reflux around the stent.

Preferably the flexible material of said stent has an external diameter in the range 1 mm to 5 mm.

More preferably the flexible material of said stent has as external diameter in the range 1.5 mm to 3 mm.

Preferably the flexible material of said stent is sof flex™, endo sof™ or ultrathane™.

The invention is further described with reference to the following figures wherein:

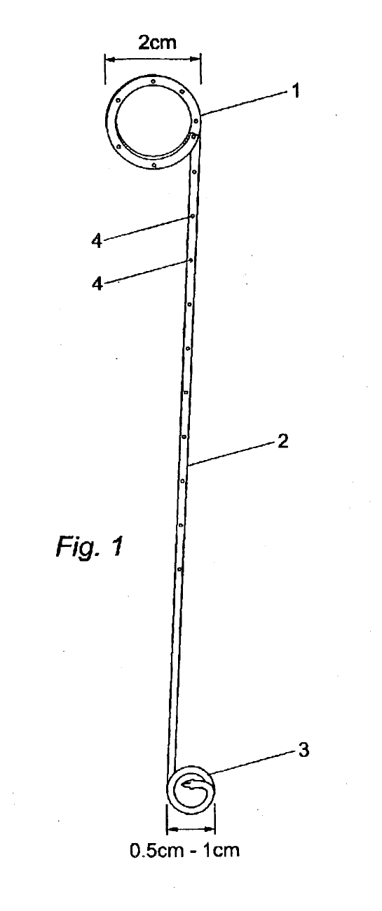

FIG. 1 illustrates a preferred embodiment of the stent

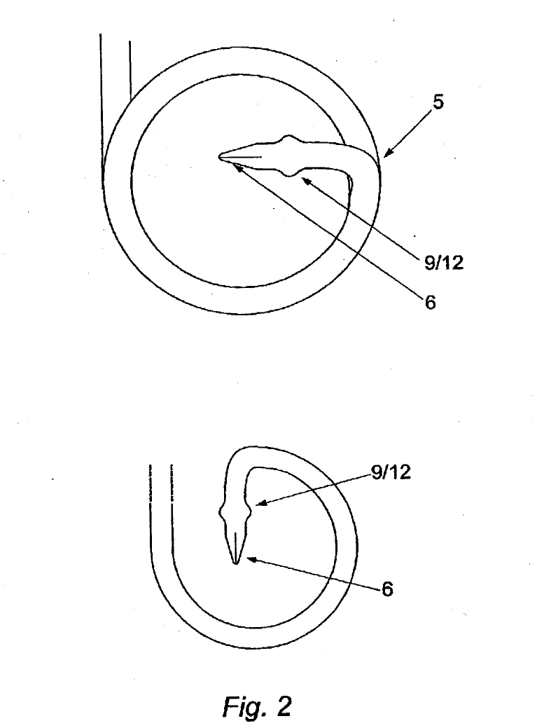

FIG. 2 illustrates the G shaped coil

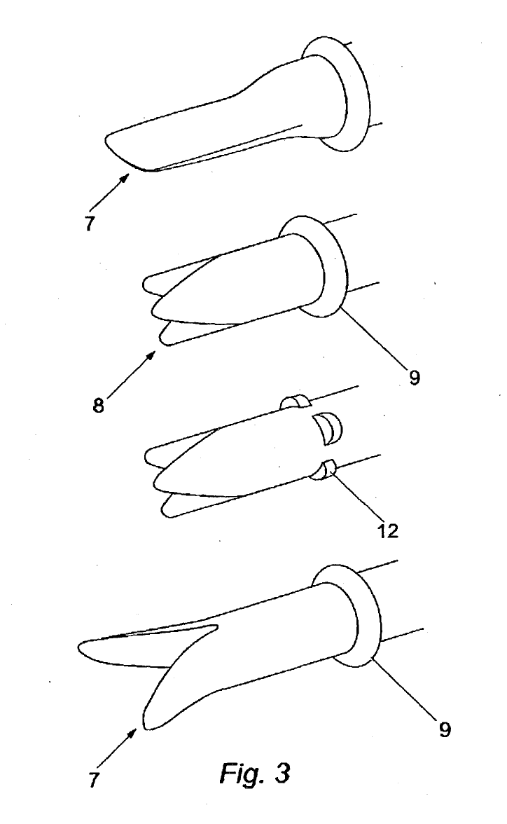

FIG. 3 illustrates the integral valve

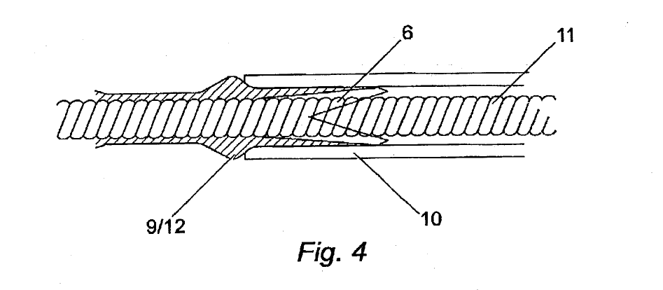

FIGS. 3 and 4 illustrate the projections against which a stent pusher can rest.

In one specific embodiment the invention provides a stent which consists of a single piece of flexible material which can be of any suitable composition in that the material forms a hollow tube such as sof flex™, endo sof™ or ultrathane™. This tube is moulded into an upper coil (1) a straight segment (2) and a lower coil (3). FIG. 1 is a representation of such a stent. A cross section of the stent is typically cylindrical but may also be modified into any shape in cross section either throughout it's whole length or in one section alone such as in the lower third alone (such as a spiral, a star shape or an oval) to facilitate drainage around the outside of the stent or alternatively to aid passage through the urinary anatomy during the placement procedure or allow accommodation to the natural contours of the urinary tract preventing reflux around the stent.

The diameter of the cylinder can be of any size but externally would be from about 1.5-3 mm (usually 1.9 mm ie. 6 French gauge) with an internal diameter of about 0.5-2.0 mm in the upper coil (1), typically 0.9 mm. This diameter can be maintained throughout the length of the whole stent.

In a further embodiment of the present invention the diameter of 6 French gauge (1.9 mm) may be only maintained for the upper two thirds of the middle segment (2). The diameter then tapers to a diameter of 1.5 mm (4.7 French gauge) in the lower third of the middle segment (2) and the lower coil (3).

In both embodiments of the present invention described above, the upper coil (1) will use about 6-15 cm of material coiled once or twice upon itself over a diameter of about 1 to 2.5 cm. It will allow significant uncoiling during placement to adjust for varying lengths of ureters in different patients.

The middle segment (2) will generally be about 22 cm long, but may be varied to the approximate length of the patients ureter and both it and the upper coil (1) will have small perforations at regular intervals (4) allowing the passage of urine from the outside to the inside of the stent and vice-versa. These perforations will stop in the lower third of the straight segment to avoid reflux in and below this area.

The lower coil (3) is made up of about 4 to 5 cm of material coiled into a smaller diameter curl of either 1.5 cm if it maintains the diameter of a 6 French gauge throughout its entirety or 1 cm if it tapers to 4.7 French gauge size. This coil will have a G-shape such that the end of the stent forms the horizontal part of the G. Representations of such a coil are shown in FIG. 2. The G-shape formation of the lower coil prevents the distal tip of the stent touching other parts of the stent and impeding the free action of the valve on this end. It also prevents the end of the stent digging into and irritating the bladder. As mentioned above, this part of the stent is of a smaller diameter (usually 1.5 mm ie. 4.7 French gauge) to reduce bladder irritation. The tip of the lower end of the stent is cut and moulded to form a valve (6,7,8), which may be of any kind, but will preferably be of a bicuspid or tricuspid type. Representations illustrating embodiments of the valve as bicuspid and tricuspid types are shown in FIG. 3. In a preferred embodiment a bicuspid valve may be provided through moulded interlay of the material comprising the stent, such that in the resting position the 2 leaflets of the valve lie flat against each other providing a seal which prevents urine passing up the stent.

In the stent whose G has diameter of 6 French gauge or 1.9 mm in size, the length of the valve will be 7 mm.

In the further embodiment of the present invention in which the diameter tapers down to 4.7 French gauge or 1.5 mm in size, the valve itself will be 5 mm long.

The valve leaflets will easily be pushed apart by urine passing down the stent or the guide wire onto which the stent is fed during placement.

Located about 3 mm behind the valve in both 6 French gauge 1.9 mm) and 4.7 French gauge (1.5 mm) is a small cuff, or four studs (12) which are again moulded out of the flexible material. This cuff (9) or four studs (12) is used for the stent pusher to rest against when placing the stent over a flexible metal guide wire, this is shown in FIG. 4.

Placement of the stent is facilitated by means of a conventional cystoscope using a conventional guide wire (11) passed through the urethra into the bladder, through the ureteric orifice up the ureter and into the renal pelvis under fluoroscopic control. The stent is fed onto the guide wire with the upper coil first and then pushed into place using a modified conventional stent pusher (10) which fits over the valve and rests against the cuff just behind the valve at the lower curl, thereby minimising trauma to the valve on insertion. Once the stent is in place the guide wire and stent pusher are removed.

Removal of the stent would be through the urethra using a cystoscope or alternatively from above either at the time of surgery on the kidney or with percutaneous retrieval devices.

1) Holes in the upper two thirds of the stent allow maximum drainage in and out of the stent to overcome any upper ureteric obstruction. The lack of perforations in the lower third and lower coil prevent reflux.

2) The tapering arrangement whereby the tube decreases in external diameter from 1.9 mm (6 French gauge) at the upper diameter to 1.5 mm (4.7 French gauge) lower diameter allows maximum drainage in the upper urinary tract and minimum irritation in the lower urinary tract.

3) The small size of the lower coil causes less bladder irritation than conventional stents.

4) The assumption of a G-shape of the lower coil ensures that the end of the stent which is normally free to dig into the bladder does not do this, thereby minimising stent induced irritation, which can itself produce unstable bladder contractions and secondary reflux of urine.

5) The advantage of the herein described valve over existing valves is that the present valve is an integral part of the stent rather than being stuck on, therefore there is virtually no risk of a piece of the stent falling off or becoming partially detached from the main body of the stent as a retained foreign body. It is also much smaller than existing polythene bag valves and should therefore cause less bladder irritation.

The present invention can be inserted into patients using a traditional procedure as described above.

1. An indwelling ureteral stent constructed of a flexible material including a hollow elongated tubular body, the hollow elongated tubular body comprising an upper end section (1), a substantially straight middle section (2) and a lower end section (3) characterised in that the tip of the lower end section forms an integral valve (6).

2. An indwelling ureteral stent as claimed in claim 1, wherein the lower section forms a closed or substantially closed loop, such that in use the tip of the lower end section (3) of the stent does not contact the bladder lining.

3. An indwelling ureteral stent as claimed in claims 1 or 2, wherein the lower end section (3) is “G” shaped.

4. An indwelling ureteral stent as claimed in any preceding claim wherein the lower section is formed into a “G” shape and where the tip of the stent assumes the horizontal portion of the G shape.

5. An indwelling ureteral stent as claimed in claim 2, wherein the lower end section (3) is spherical in shape.

6. An indwelling ureteral stent as claimed in any of the preceding claims wherein the lower end section (3) has a diameter of between 0.5 to 2 cm.

7. An indwelling ureteral stent as claimed in any of the preceding claims wherein the upper section (1) comprises a coil, the coil being formed of between 6 to 15 cm of material coiled once or twice upon itself, the coil having a resulting diameter of between 1 to 2.5 cm.

8. An indwelling ureteral stent as claimed in claims 1 to 7, wherein the valve is a bicuspid valve having two leaflets (7) or a tricuspid valve having three leaflets (8).

9. An indwelling ureteral stent as claimed in claims 1 to 8, wherein the valve is provided through the moulded interlay of the flexible material such that in the closed position the leaflets of the valve lie flat against each other providing a seal which prevents fluid passing into the stent.

10. An indwelling ureteral stent as claimed in any preceding claim wherein the flexible material decreases in external diameter from the upper end section (1) to the lower end section (3).

11. An indwelling ureteral stent as claimed in claim 10, wherein the flexible material is tapered from the upper end section (1) to the lower end section (3), such that the lower third of the middle section (2) and the totality of the lower section (3) are of a reduced diameter to that of the upper end section (1).

12. An indwelling ureteral stent as claimed in any preceding claim wherein the stent further comprises at least one projection (12) against which a stent pusher may rest.

13. An indwelling ureteral stent as claimed in claim 12 wherein the at least one projection forms a cuff (9).

14. An indwelling ureteral stent as claimed in claim 12 wherein the at least one projection consist of a plurality of studs.

15. An indwelling ureteral stent as claimed in any preceding claim wherein the stent is constructed of a flexible material.

16. An indwelling ureteral stent as claimed in claim 15, wherein the flexible material includes any composition which forms a hollow tube.

17. An indwelling ureteral stent as claimed in claim 15 wherein the flexible material has a cylindrical cross section.

18. An indwelling ureteral stent as claimed in claim 15 wherein the flexible material has a spiral, star or oval shaped cross section.

19. An indwelling ureteral stent as claimed in any of claims 15 to 18 wherein the flexible material of said stent has an external diameter in the range 1 mm to 5 mm.

20. An indwelling ureteral stent as claimed in any of claims 15 to 18 wherein the flexible material of said stent has an external diameter of between 1.5 mm to 3 mm.

21. An indwelling ureteral stent as claimed in any of claims 15 to 20 wherein the flexible material is sof flex™, endo sof™ or ultrathane™.
